(19) United States
(12) Patent Application Publication
(10) Pub. No.: US 2010/0292739 A1
(43) Pub. Date: Nov. 18, 2010

(54) Bone Screws With Improved Locking Mechanisms

(75) Inventor: Frank J. Schwab, New York, NY (US)

Correspondence Address: MEDTRONIC;Attn: Noreen Johnson - IP Legal Department, 2600 Sofamor Danek Drive, MEMPHIS, TN, 38132, US

(73) Assignee: WARSAW ORTHOPEDIC, INC., Warsaw, IN (US)

(21) Appl. No.: 12/466,423

(22) Filed: May 15, 2009

(51) Int. Cl. A61B 17/86

(52) U.S. Cl. 606/305; 606/301

(57) ABSTRACT

Screw mechanisms that securely connect with and contain an elongated support structure are disclosed. Some of these have locking mechanisms as alternatives to conventional set screws. Some of these use ratcheting systems for securing the elongated support structure. Some of these are multi-axial systems. Some of these are alternatives to conventional systems.

### TECHNICAL FIELD

This disclosure relates in general to screw mechanisms for securing a ligament, tether, rod, and other supporting structure to bone.

### BACKGROUND

Several techniques, systems, and supporting structures have been developed for correcting and stabilizing the spine. Some systems use supporting structures like a rod, a tether, a ligament, or others disposed longitudinally along a length of the spine or vertebral column. In accordance with such a system, the supporting structure is engaged to various vertebrae along a length of the spinal column by way of a number of fixation elements. A variety of fixation elements are configured to engage specific portions of the vertebra. For instance, one such fixation element is a hook that is configured to engage the laminae of the vertebra. Another very prevalent fixation element is a spinal bone screw which can be threaded into various aspects of the vertebral bone or pelvis. For example, a plurality of spinal bone screws can be threaded into a portion of several vertebral bodies and the sacrum, such as, for example, the pedicles of these vertebrae. The supporting structures can then be affixed to these spinal bone screws to apply corrective and stabilizing forces to the spine.

Conventional bone screws can be improved to more easily implant or secure the supporting structures, more easily align the supporting structures, or provide other benefits.

The present disclosure overcomes one or more shortcomings in the art.

### SUMMARY

The present disclosure is directed to improved screw mechanisms that securely connect with and contain an elongated support structure. Some of these have locking mechanisms as alternatives to conventional set screws. Some of these use ratcheting systems for securing the elongated support structure. Some of these are multi-axial systems. Some of these are alternatives to conventional systems.

In one exemplary aspect, a screw mechanism for connection with an elongated supporting structure is disclosed. In exemplary aspect disclosed herein the mechanism includes a threaded anchor portion configured to penetrate bone tissue and includes an outer receiver attached to the threaded anchor portion. The outer receiver is formed with first and second extending legs and has a transverse passage therethrough. The passage has inner sidewalls. The mechanism also includes an inner receiver receivable into the outer receiver and having outer sidewalls. The outer sidewalls of the inner receiver and the inner sidewalls of the outer receiver passage have articulating or deformable connectors formed thereon. These connectors cooperate to permit the inner receiver to articulate relative to the outer receiver. The inner receiver is formed with a first longitudinally extending opening and has a second transverse opening therethrough sized and shaped to receive the stabilizing supporting structure. The inner receiver is deformable from an unlocked condition where the inner receiver is not frictionally engaged with the U-shaped inner surface in a manner that prevents articulation of the inner receiver relative to the outer receiver to a locked condition where the inner receiver is frictionally engaged with the U-shaped inner surface in a manner that prevents articulation of the inner receiver relative to the outer receiver. The mechanism also includes a locking mechanism receivable into the first longitudinally extending opening and configured to apply a driving force that deforms the inner receiver from the unlocked condition to the locked condition.

In another exemplary aspect, the screw mechanism includes an outer receiver formed with first and second extending legs forming a U-shaped inner surface, the U-shaped surface having a bottom surface and inner sidewalls. An inner receiver is receivable into the outer receiver. The inner receiver has outer sidewalls. The inner receiver is formed with a first longitudinally extending opening having threads on the inner circumference and having a second transverse opening therethrough sized and shaped to receive the elongated supporting structure. The inner receiver has legs deformable from an unlocked condition where the legs are not frictionally engaged with the U-shaped inner surface in a manner that prevents articulation of the inner receiver relative to the outer receiver to a locked condition where the legs are frictionally engaged with the U-shaped inner surface in a manner that prevents articulation of the inner receiver relative to the outer receiver. A locking mechanism is receivable into the first longitudinally extending opening and configured to apply a driving force that deforms the inner receiver from the unlocked condition to the locked condition.

In another exemplary aspect, the screw mechanism includes an outer receiver formed with first and second extending legs and having a transverse passage therethrough, the passage having inner sidewalls. An inner receiver is receivable into the outer receiver and has outer sidewalls and is configured to articulate relative to the outer receiver. The inner receiver has a first longitudinally extending opening and a second transverse opening. The second transverse opening is sized and shaped to receive the stabilizing supporting structure. The first longitudinal opening is in communication with the second transverse opening. The inner receiver is deformable from an unlocked condition where the inner receiver is not frictionally engaged with the U-shaped inner surface in a manner that prevents articulation of the inner receiver relative to the outer receiver to a locked condition where the inner receiver is frictionally engaged with the U-shaped inner surface in a manner that prevents articulation of the inner receiver relative to the outer receiver.

Further aspects, forms, embodiments, objects, features, benefits, and advantages of the present invention shall become apparent from the detailed drawings and descriptions provided herein.

### BRIEF DESCRIPTION OF THE DRAWINGS

FIGS. 1 and 2 are illustrations of a first embodiment of an exemplary bone screw system for securing an elongated supporting structure in accordance with one or more aspects of the present disclosure.

FIGS. 3 and 4A-4C are illustrations of a second embodiment of an exemplary bone screw system in accordance with one or more aspects of the present disclosure.

FIGS. 5A-5C are illustrations of a third embodiment of an exemplary bone screw in accordance with one or more aspects of the present disclosure.

FIGS. 6-8, 9A, 9B, and 10A-10C are illustrations of a fourth embodiment of an exemplary bone screw in accordance with one or more aspects of the present disclosure.

FIG. 11 is an illustration of an exemplary top clamp usable on a bone screw in accordance with one or more aspects of the present disclosure.

FIGS. 12, 13A, 13B, and 14 are illustrations of a fifth embodiment of an exemplary bone screw in accordance with one or more aspects of the present disclosure.

FIGS. 15 and 16 are illustrations of a sixth embodiment of an exemplary bone screw in accordance with one or more aspects of the present disclosure.

FIGS. 17A and 17B are illustrations of a seventh embodiment of an exemplary bone screw in accordance with one or more aspects of the present disclosure.

FIGS. 18A and 18B are illustrations of an eighth embodiment of an exemplary bone screw in accordance with one or more aspects of the present disclosure.

FIGS. 19-21 are illustrations of a ninth embodiment of an exemplary bone screw and an associated assembly tool in accordance with one or more aspects of the present disclosure.

FIGS. 22A and 22B are illustrations of a tenth embodiment of an exemplary bone screw in accordance with one or more aspects of the present disclosure.

FIGS. 23-26 are illustrations of an eleventh embodiment of an exemplary bone screw in accordance with one or more aspects of the present disclosure.

FIG. 27 is an illustration of a twelfth embodiment of an exemplary bone screw in accordance with one or more aspects of the present disclosure.

FIGS. 28-30 are illustrations of a thirteenth embodiment of an exemplary bone screw in accordance with one or more aspects of the present disclosure.

FIG. 31 is an illustration of an alternative shell ring usable with the exemplary bone screw of FIGS. 28-30.

### DETAILED DESCRIPTION

For the purposes of promoting an understanding of the principles of the invention, reference will now be made to the embodiments, or examples, illustrated in the drawings and specific language will be used to describe the same. It will nevertheless be understood that no limitation of the scope of the invention is thereby intended. Any alterations and further modifications in the described embodiments, and any further applications of the principles of the invention as described herein are contemplated as would normally occur to one skilled in the art to which the invention relates.

The embodiments disclosed herein are directed toward systems for more predictably and reliably securing elongated supporting structure, such as ligaments, tethers, and rods, for example, to a bone screw. Furthermore, many of these embodiments simplify the procedure for securing the elongated supporting structure within a bone screw through controlled compression between the screw and the elongated supporting structure. Some of these use a locking mechanism having a preliminary locking condition that loosely secures the elongated supporting structure in place and a final locking condition that more securely holds the elongated supporting structure in place. In addition, some of these embodiments do not require threading of a set screw to secure the elongated supporting structure in place. This reduces the difficulties of ligament reduction and perfect set screw alignment.

Various embodiments are described as being used with a ligament, a tether, or a rod. However, these embodiments are not limited to being used with the specific type of elongated supporting structures described. It is contemplated that any of the embodiments described herein may be used with any elongated supporting structure, including but not limited to ligaments, rods, tethers, bands, and natural or synthetic devices, known and unknown. In addition, these embodiments are described as being connected to threaded anchor portions. It is contemplated that hooks or other bone connecting mechanisms can be used in place of the threaded anchors.

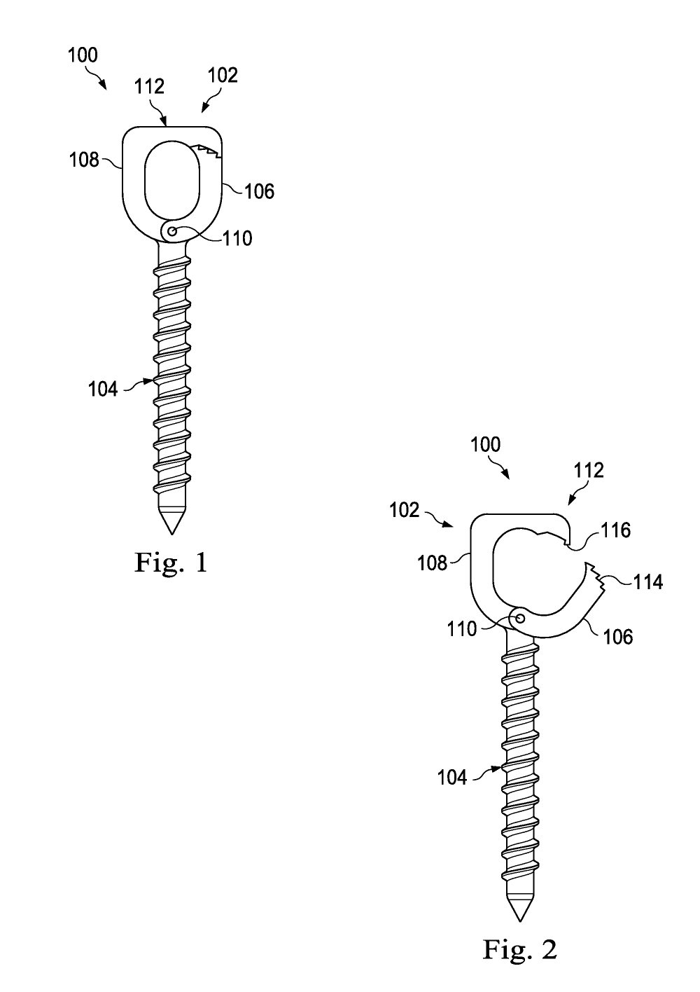

FIG. 1 shows an exemplary screw mechanism 100. The screw mechanism 100 comprises a receiver 102 and a threaded portion 104. In this exemplary embodiment the receiver 102 comprises a first arm 106 and a second arm 108. Together these arms receive and secure an elongated supporting structure like a ligament in place. In this embodiment, one or both of the first and second arms 106, 108 pivot relative to the other about a swivel mechanism 110. In some embodiments, the swivel connection is a hinge or pin connection or deformable linkage. The swivel mechanism 110 in this embodiment is a pin extending through a base portion of the first arm 106 and the second arm 108, thereby pivotably connecting the arms together. Accordingly, the first arm 106 may pivot relative to the second arm 108.

In this embodiment, as is apparent in FIG. 2, the first arm 106 is movable about the swivel mechanism 110 while the second arm 108 is rigidly fixed relative to the threaded anchor portion 104. In another exemplary embodiment, the second arm 108 is pivotable relative to the threaded anchor portion 104 and the first arm 106 is fixed relative to the threaded anchor portion 104. In another embodiment, both the first and second arms 106, 108 pivot relative to each other and the threaded anchor portion 104.

The screw mechanism 100 includes a locking system 112 for placing the screw mechanism in a preliminary locking condition and in a final locking condition to fixedly secure a ligament within the receiver 102 without the use of a set screw. In this embodiment, the locking system 112 includes a plurality of teeth 114, which in this embodiment are formed on the first arm 106 and at least one ratcheting engagement tooth 116 formed on the second arm 108. When a ligament or other longitudinal linkage member is introduced into the receiver 102 the movable first or second arm 106, 108, depending upon the embodiment, will move until the ratcheting engagement tooth 116 engages the teeth 114 of the opposing arm. As the ratcheting engagement tooth 116 ratchets along the teeth 114 the ligament is in a preliminary locking condition within the receiver 102. Accordingly, it can be manipulated within the receiver such that it may slid axially through the receiver or otherwise be manipulated by the service provider for final placement, but it cannot be removed from the receiver. When the ligament or other type of longitudinal linkage member is properly located, the service provider places the locking system 112 in a final locking condition by further compressing the first and second arms 106 and 108 together such that the ratcheting engagement tooth 116 further engages the additional teeth 114, decreasing the inner receiver space and compressing upon the ligament or longitudinal linkage member.

In this embodiment, the teeth 114 are shown on first arm 106 and an engagement tooth 116 is shown on the second arm 108. Nevertheless, it should be apparent that the teeth 114 may optionally be disposed on the second arm 108 and the engagement tooth 116 may be disposed on the first arm 106. Also, in the embodiment shown, the second arm 108 extends across the top portion of the screw mechanism 100. In other embodiments, both the first and second arms 106, 108 are shaped so that the locking mechanism is disposed directly above a ligament when the screw mechanism 100 is finally locked on a ligament. Such embodiments accommodate placing the ligament into the receiver from the top, rather than from the side. Other arrangements are also contemplated.

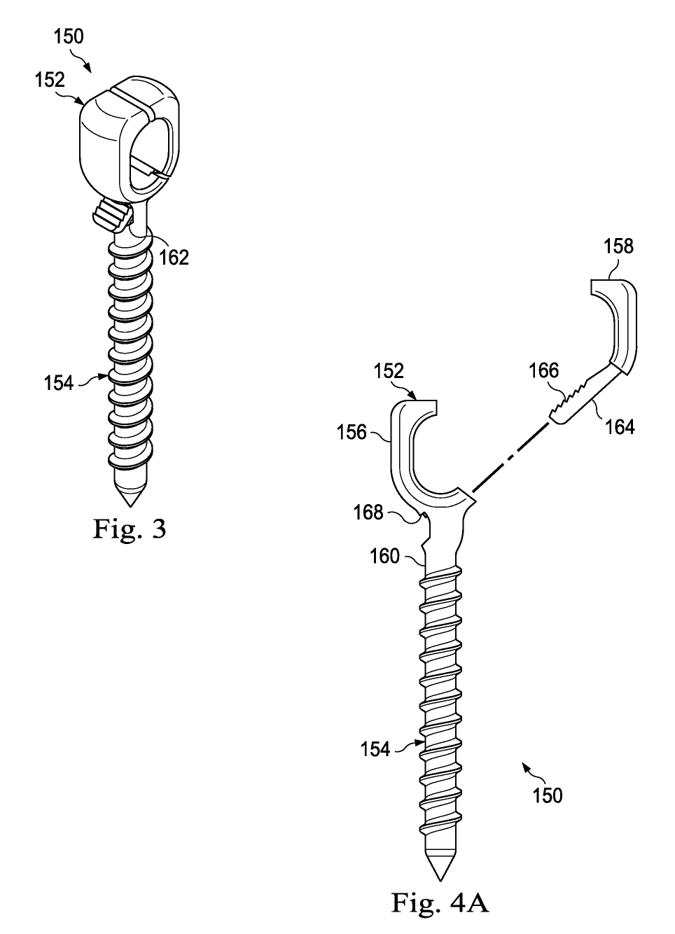

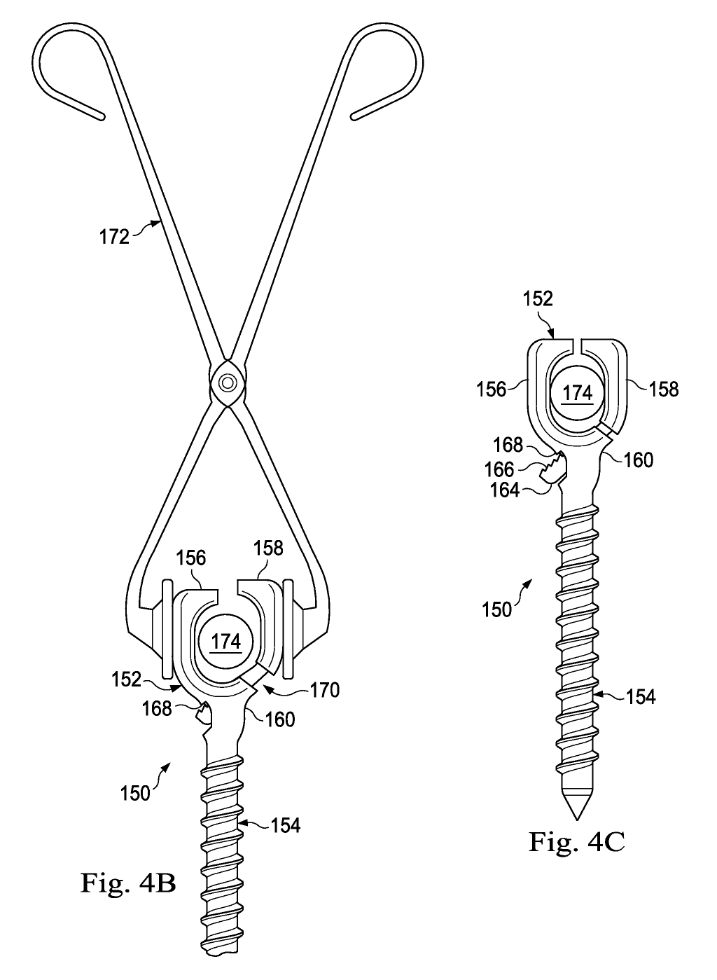

FIGS. 3 and 4A-4C show another embodiment of a screw mechanism, referenced herein by the numeral 150. Like the screw mechanism 100 described above, the screw mechanism 150 includes a receiver 152 and a threaded anchor portion 154. The receiver 152 comprises a first arm 156 and a second arm 158. Together the first and second arms 156, 158 form a ligament receiving opening for receiving the ligament. In this embodiment, a base portion 160 adjacent the threaded anchor portion 154 and the first arm 156 includes a passage or aperture 162 (best seen in FIG. 3) that receives a leg 164 of the second arm 158. In this embodiment, the leg 164 has a series of teeth 166 formed thereon. The aperture 162 likewise has at least one tooth 168 formed thereon for engaging and ratcheting with the teeth 166 in a manner similar of that discussed above relative to the engagement tooth 116 and the tooth 114 of FIGS. 1 and 2. Accordingly, these teeth form a locking system 170 that comprises the teeth 166, the aperture 162, and a locking engagement tooth associated with the aperture 162 and the first arm 166.

Accordingly, by inserting the leg 164 of the second arm 158 into the aperture 162 until the teeth 166, 168 engage, the receiver 152 may be placed in a preliminary locking condition that prevents removal of the ligament from the receiver, but still allows manipulation of the ligament in the receiver. Further advancement of the leg 164 through the aperture 162 ratchets the teeth 166, 168 places the receiver 152 in a final locking condition, securing the ligament in place. FIG. 4B shows that further advancement may be obtained using an insertion tool 172 that applies lateral loading on the receiver 152 to compress the arms 156, 158 into frictional engagement with a ligament 174. FIG. 4C shows the ligament 174 frictionally secured in place.

All of the screw mechanisms disclosed herein may be formed of any material suitable. In some embodiments, portions of the screw mechanisms are formed of elastic materials suitable for ratcheting and interlocking teeth in the manner described. These materials may include for example, any member of the polyaryletherketone (PAEK) family such as polyetheretherketone (PEEK), carbon-reinforced PEEK, or polyetherketoneketone (PEKK); polysulfone; polyetherimide; polyimide; ultra-high molecular weight polyethylene (UHMWPE); and/or cross-linked UHMWPE. In addition, it may include materials such as cobalt-chromium alloys, titanium alloys, nickel titanium alloys, and/or stainless steel alloys. Shape memory materials having pre-formed memory structures also may be used. Other polymers and metals also are contemplated whether known or unknown at this time.

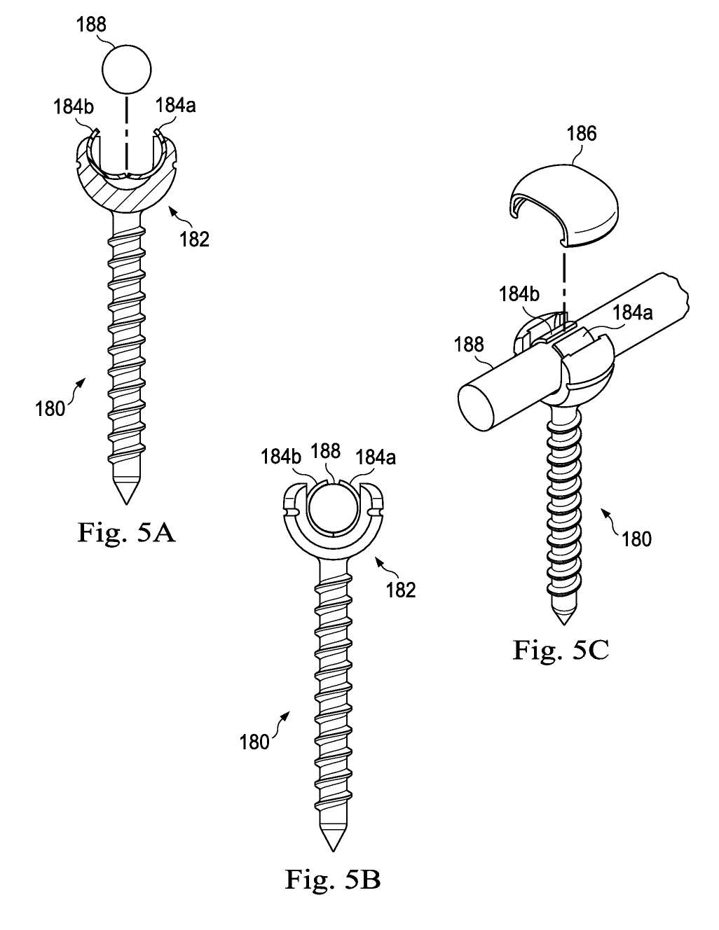

FIGS. 5A-5C show an additional embodiment of a screw mechanism reference herein by the numeral 180. The screw mechanism 180 includes a receiver 182, swiveling side blockers 184a and 184b, and a staple 186 (shown in FIG. 5C). In this embodiment the swiveling side blockers 184 are formed as semi-cylindrical shells that together cooperate to extend at least partially about the external surface of the ligament. FIG. 5A shows the swiveling side blockers 184 in an open condition ready to receive a ligament 188.

As will become apparent, the side blockers 184 and the staple 186 form a locking system with a preliminary locking condition and a final locking condition. FIG. 5B shows the ligament 188 after being contained by the swiveling side blockers 184a and 184b. As can be seen, as the ligament 188 moves downward between the side blockers 184, they pivot to enclose and provisionally lock the ligament 188 within the receiver 182. In some embodiments, the side blockers in this condition cannot be removed from the receiver 182. Accordingly, in this condition, the screw mechanism 180 is a preliminary locking condition.

Referring to FIG. 5C, the staple 186 cooperates with the side blockers 184 of the receiver 102 to place the screw mechanism 180 in a final locking condition. In this embodiment, the staple 186 extends about an exterior of the receiver 182 and applies compression loading upon the receiver 182. In some embodiments, the receiver sidewalls deform based upon the staple compression to apply loading onto the swiveling side blockers to frictionally lock the blockers relative to the receiver. In other embodiments, the staple 186 presses directly on the ligament and/or blockers to apply a compressive load against the bottom of the U-shape in the receiver 182.

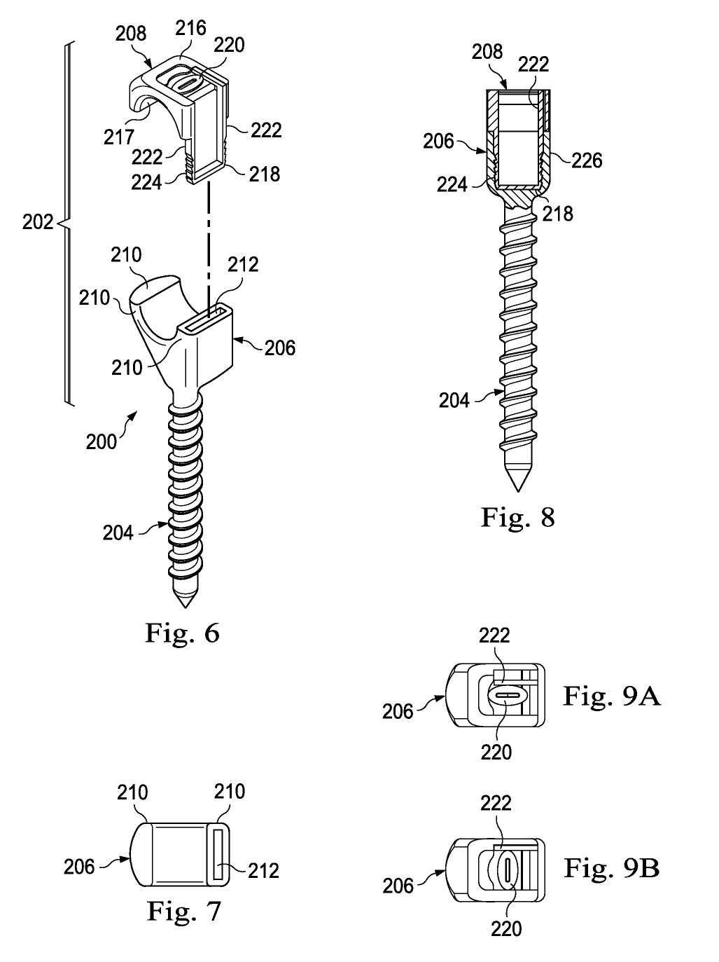

FIGS. 6 through 10 show another embodiment of a screw mechanism referenced herein as 200. The screw mechanism 200 comprises a receiver 202 and a threaded anchor portion 204. Here, the receiver 202 includes a tulip head forming a bottom clamp 206 and includes top clamp 208. The bottom clamp 206 includes first and second extending sides 210 that form a transverse opening for receiving the ligament. One of the sides 210 includes a slot 212 formed therein for receiving a portion of the top clamp 208.

The top clamp 208 comprises a frame 216, a ratchet clip 218 and a cap 220. In this embodiment, the ratchet clip 218 forms a locking system having a preliminary locking configuration and a final locking configuration for securing an elongated supporting structure or ligament.

The frame 216 includes a curved inner surface 217 for interfacing with and securing the ligament between the top and bottom clamps 208, 206. The ratchet clip 218 comprises two ratchet arms 222 shaped and configured to be received within the slot 212 of the bottom clamp 206. The ratchet arms 222 have outwardly facing teeth 224 formed thereon for interlocking with corresponding teeth within the slot 212, described below.

In one embodiment, at least one of the arms 222 is compliant and elastically deformable. In this embodiment, the arm 222 can be biased toward an engaged position with the teeth in the slot 212, yet also can elastically deform to disengage the ratchet clip 218 from the teeth in the slot 212.

FIG. 7 shows a top view of the bottom clamp 206 with the slot 212. FIG. 8 shows a partial cross-section taken through the slot 212 with the ratchet clip 218 of the top clamp 208 in place within the slot 212. In this cross-section, the engagement teeth 224 of the ratchet clip 218 are shown engaged with corresponding teeth 226 formed in the slot 212 of the bottom clamp 206. The compliant nature of the arm 222 permits the upper clamp 208 to be received into the slot 212 and such that the teeth 222 engage the teeth 224 in a preliminary locking condition to secure a ligament within the screw mechanism 200.

A cap 220 cooperates with the ratchet clip 218 to prevent or reduce the ability of the compliant ratchet arms to elastically deform. Accordingly, the cap 220 and the teeth 224, 226 create a locking system with preliminary and final locking conditions. Referring to FIGS. 9A and 9B, the cap 220 is configured to pivot about an axis to permit the top clamp to be provisionally secured to the bottom clamp 206 and also to lock the top clamp 208 to the bottom clamp 206. As can be seen in FIG. 9, the cap 220 has a noncircular shape. During rotation of the cap, its outer perimeter acts as a cam against one of the locking arms 222 of the ratchet clip 218. For example, referring to FIG. 9A, when the cap 220 is aligned primarily in a longitudinal direction as shown, the arm 222 may elastically deflect to permit the ratchet clip 218 to move up or down within the slot 212, thereby permitting the clamps 206, 208 to open and close relative to each other. As can be seen in FIG. 9B, rotating the cap 220 90° displaces the arm 222 and restricts movement of the arm 222. Accordingly, the teeth 224 on the arm 222 cannot disengage the teeth 226 in the slot 212 in the bottom clamp 206. This is a final locking position.

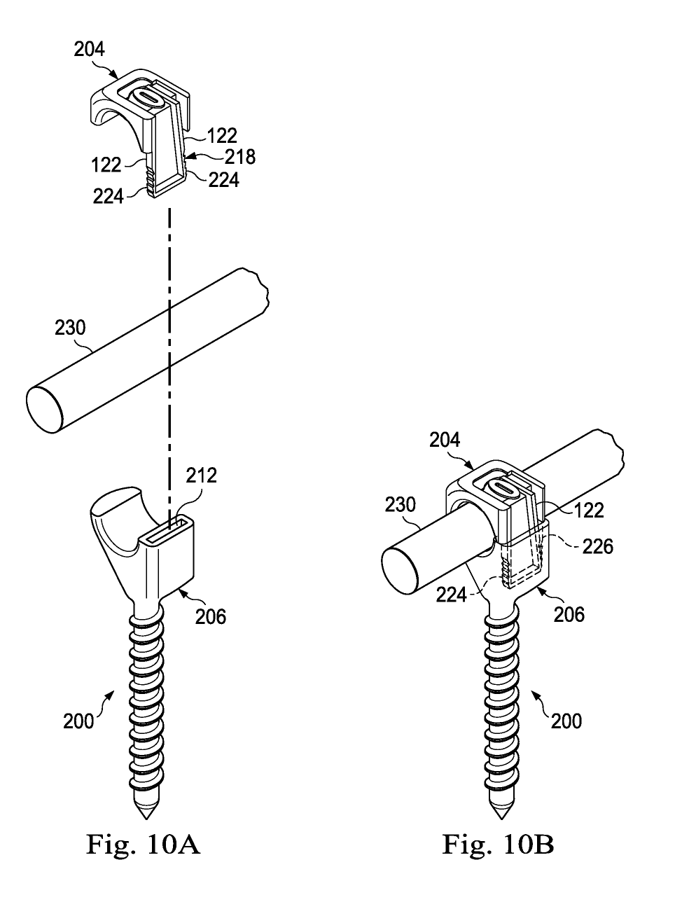

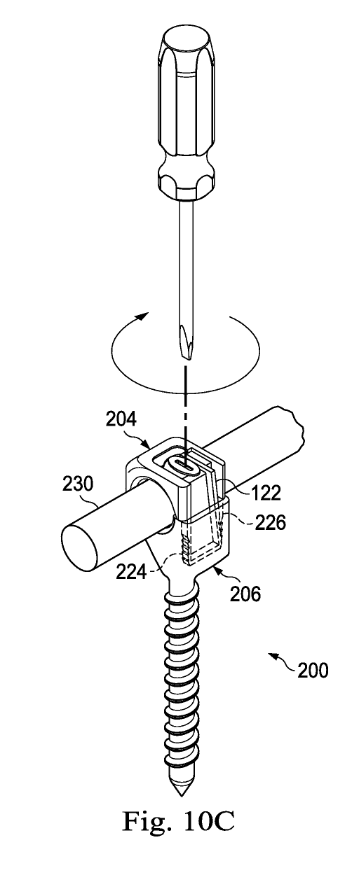

FIGS. 10A-10C show a process for securing a ligament 230 within the screw mechanism 200. Referring first to FIG. 10A, a ligament 230 may be placed in the tulip shaped bottom clamp 206. The top clamp 208 may be placed over the ligament 230 so that the ratchet clip 218 enters the slot 212. The externally facing teeth 224 on the arms 222 engage the internally facing teeth 226 within the slot 212 of the bottom clip 206. Because the arm 222 is elastically deformable, the teeth 224 can engage and disengage the teeth 226 to permit the top clamp 206 to be moved up or down to place the screw mechanism in a preliminary locked condition about the ligament.

In some embodiments, the top clamp 206 is connected within the slot 212 prior to introducing the ligament 230. In these embodiments, the receiver 204 is a side-loading receiver. After placing the ligament 230 between the clamps, the top clamp is pressed downwardly to secure the ligament in the preliminary locked condition.

Once the ligament is properly positioned relative to the spine, the cap 220 may be rotated 90° by a locking tool to drive or splay the ratchet arms 222 apart such that the teeth 224 engage the teeth 226, placing the mechanism in a final locking condition, as shown in FIG. 10C.

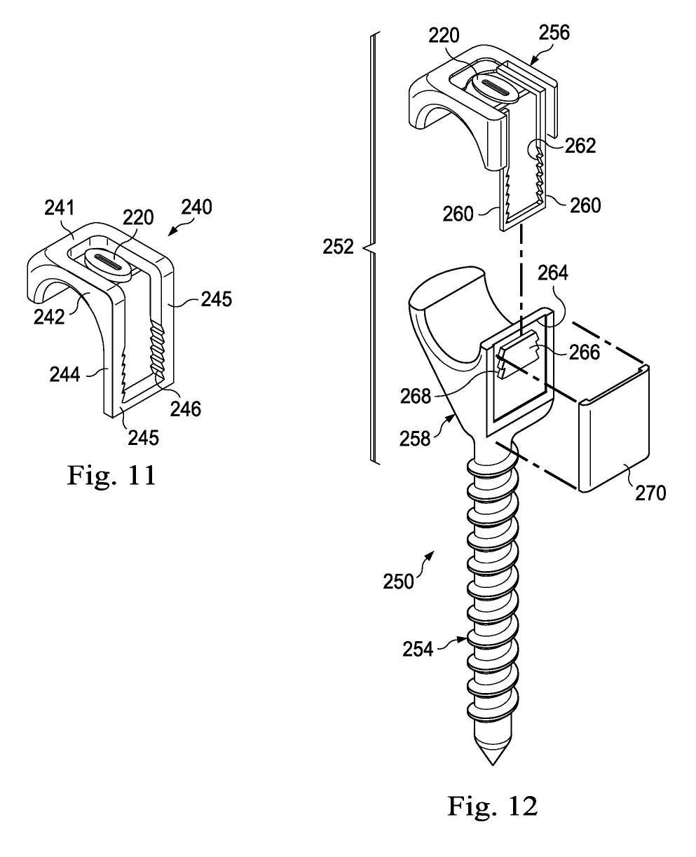

FIG. 11 illustrates an alternative embodiment of a top clamp, referenced herein as 240. The top clamp 240 includes a frame 241 that cooperates with the cap 220. Here however, the frame 241 has a top portion 242 and a bottom portion 244. The bottom portion 244 includes two legs 245 with teeth 246 extending therefrom. Here, the teeth 246 face inwardly. Accordingly, the top clamp 240 may be used to engage with a bottom clamp having outwardly facing teeth that interlock with the teeth 246. Rotating the cap 220 against the frame 241 elastically expands the frame and separates the inwardly facing teeth.

FIG. 12 discloses another exemplary embodiment of a screwing mechanism referenced herein by the numeral 250. This includes a receiver 252 and a threaded anchor portion 254. The receiver 252 includes a top clamp 256 and a tulip shaped bottom clamp 258 similar in some respects to the bottom clamp 206 described above. Like the top clamp 208 described above, the top clamp 256 includes arms 260 that are elastically deformable between a preliminary locked condition and a final locking condition. In this embodiment, teeth 262 on the arms 260 face inwardly. The cap 220 manipulates the arms 260 as described above between the preliminary locked and the final locked conditions.

In this embodiment the lower clamp 258 is formed with the slot 264 through which the arms 260 of the upper clamp 256 may be introduced. A protrusion 266 having externally facing teeth 268 is disposed within the slot 264. The teeth 268 are shaped and configured to interlock with the teeth 262 of the upper clamp 256. In this embodiment, in order to provide simple assembly, the bottom clamp 258 includes a casing cover 270. The casing cover 270 cooperates with and forms a part of the bottom clamp 258 to form the slot 264. It also reduces a chance of lateral movement of the arms 260 relative to the lower clamp 258.

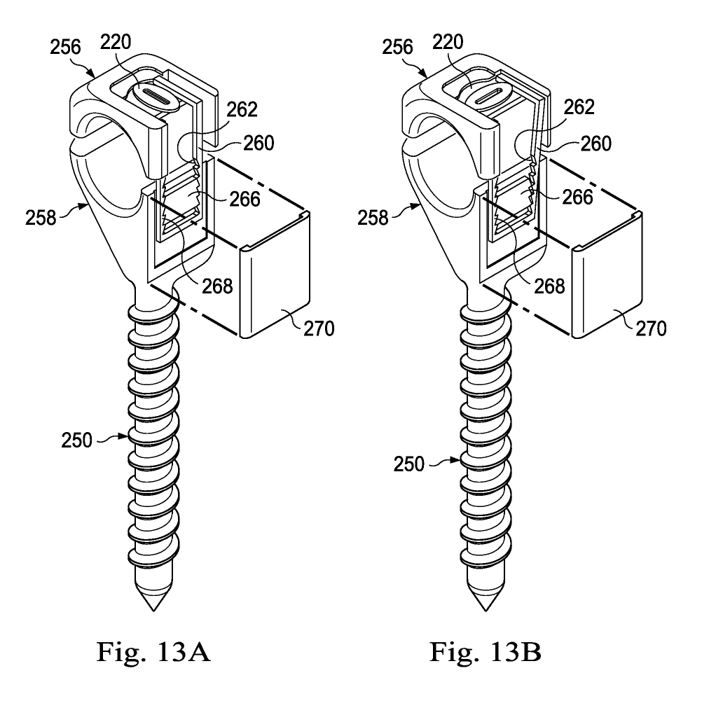

FIGS. 13A and 13B show the screw mechanism 250 in a locked condition and in an unlocked condition. Referring first to FIG. 13A, the top clamp 256 may be placed so that the arms 260 extend on either side of the protrusion 266. The teeth 262 interlock with the teeth 268 as shown in FIG. 13A. Because the tooth 262 and teeth 268 are sawtooth shaped, the upper clamp 256 may be advanced downwardly to apply compression loading against an elongated support structure that may be contained between the clamps. In so doing the arms 260 elastically deformed to accommodate the locking and interlocking teeth. Accordingly, when the top and bottom clamps are spaced apart and loosely holding a ligament in place, the clamps may be considered to be in a preliminary locked position. However, when the ligament is properly located as desired, the top clamp 256 may be forced downwardly to lock the ligament in place. In order to unlock the top and bottom clamps 256, 258, the cap 220 may be turned to forcibly displace one or both of the arms 260 as shown in FIG. 13B. In so doing, the teeth 262 disengage from the teeth 268. With the teeth disengaged, the upper clamp 256 may be moved upwardly or downwardly relative to the lower clamp.

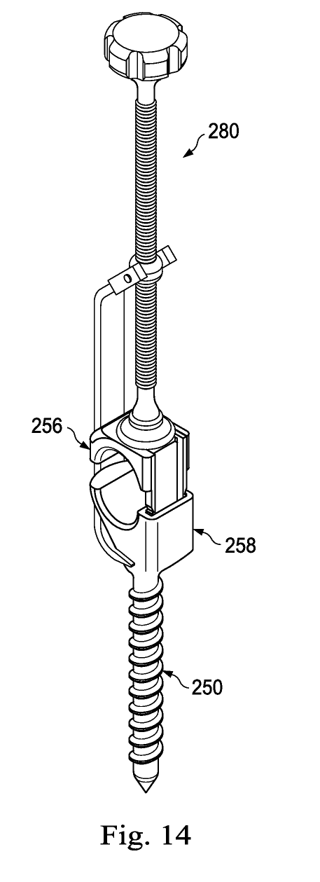

FIG. 14 shows an exemplary application tool 280 for implanting and operating the screw mechanism 250. In this case the implantation tool 280 comprises a pressure generator 280, that may be turned to drive the upper clamp downwardly relative to the lower clamp so that the ratcheting teeth on the top and bottom clamps engage. The screw mechanisms 200 and 250, as well as other embodiments disclosed herein, may be assembled intra-operatively.

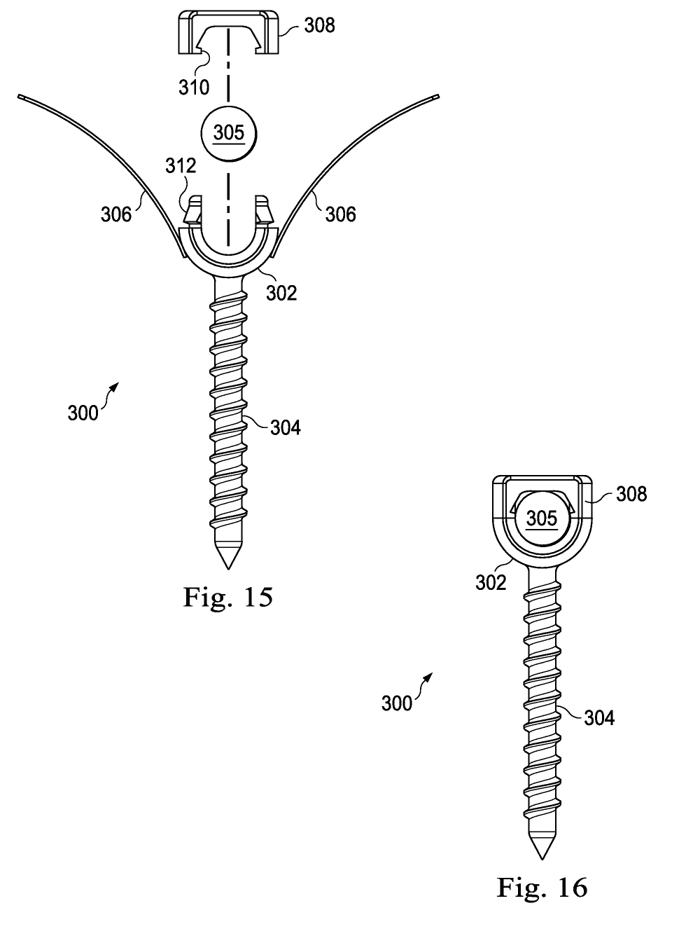

FIG. 15 shows an additional embodiment of a screw mechanism referenced herein by the numeral 300. The screw mechanism includes a receiver 302 and a threaded anchor portion 304. As with the other embodiments disclosed herein, the receiver 302 is tulip-shaped, having two legs forming a transverse opening through which the ligament 305 extends. In this embodiment, extenders 306 extend from opposing lateral sides of the receiver 302. These displace and secure tissue from overlapping the receiver 102 thereby providing access to the receiver 102 for the ligament 305, as shown in FIG. 15.

In this embodiment, the locking system comprises a staple 308 that may be received over the receiver 302 to secure the ligament in place and create a closed eyelet. In this embodiment, the staple 308 may be formed of an elastically deformable material and/or the receiver 302 may be formed of an elastically deformable material such that the staple 308 may be snap fitted onto the receiver 302. The embodiment shown includes a single tooth 310 on the staple 308 that may be received into a channel 312 formed about the receiver 302. Although only one tooth 310 and one receiver channel 312 are shown, it would be apparent that additional teeth and receiver channels may also be used in order to provide a preliminary locking condition that would still permit the ligament 305 to be moved relative to the receiver 302 and a final locking condition that securely locks the ligament 305 to the screw mechanism 100. In such an embodiment, to place the screw mechanism in the final locking condition, the staple would be further advanced over the receiver in a ratcheting manner. FIG. 16 shows one example where the receiver 302 and the staple 308 cooperate to secure the ligament 305 in place. Once the ligament is secured in the receiver 302, the extenders 306 may be removed from the screw mechanism.

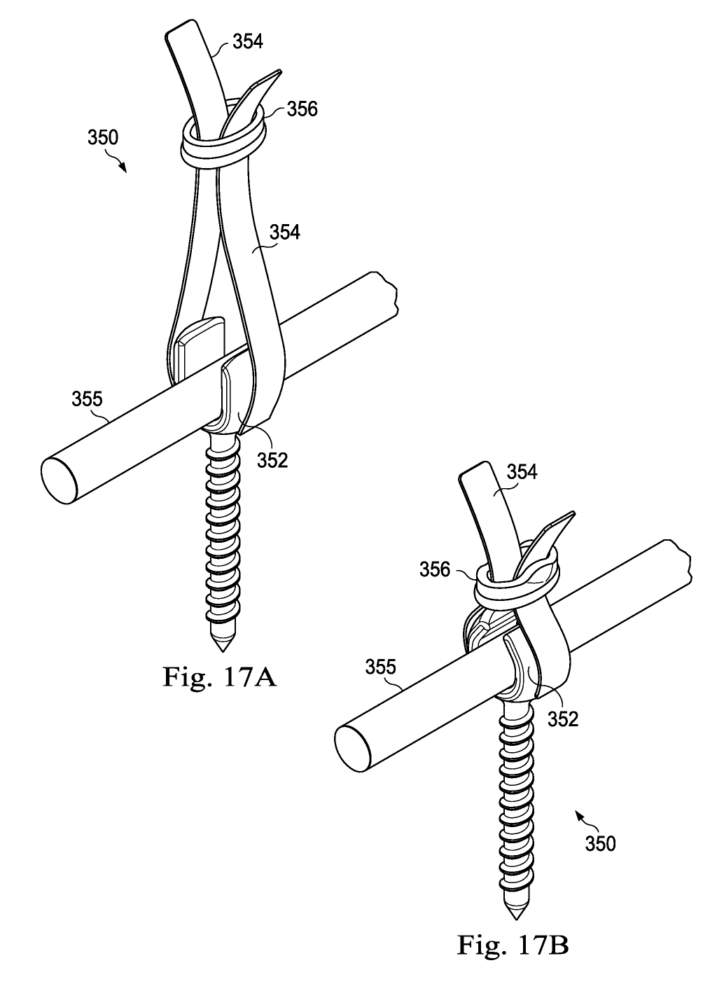

FIGS. 17A and 17B show an additional embodiment of a screw mechanism 350 for locking a ligament 355 in place. The screw mechanism 350 includes a receiver 352 and extenders 354 extending from lateral sides of the receiver 352. Accordingly, as explained above, the extenders 354 may be used to displace and hold back soft tissue so that a medical service provider has direct access to the receiver 352 for placement of a ligament 355. However, once the ligament is placed, a locking system is used to secure a ligament 355 within the receiver 352. In this case, the locking system comprises a crimpable sleeve 356 that slips over the exterior surfaces of the extenders 354 in the manner shown in FIG. 17A. These extenders may then be used to lock the ligament in place. Alternate blocking mechanisms can be envisioned such as revisable blockers, screw-based blocking mechanisms and revisable or removable crimps. For example, polymer and metal crimps may be able to be snapped into place and also may be forcibly removable to offer a revisable connection.

Referring now to FIG. 17B, the crimpable sleeve 356 may be slid along the extenders 354 until it is adjacent to the receiver 352. In this state, the device is in a preliminary locking condition because the ligament 350 is provisionally locked within the screw mechanism 350. Once the ligament is located as desired, the crimpable sleeve 356 may be further advanced over the extenders to securely hold the ligament 355 within the receiver. In the embodiment shown, the receiver 352 is formed of a deformable or compliant material that may be deformed by the extenders and crimpable sleeve 356. Accordingly, the receiver 352 may be wrapped around the ligament in the final locking condition. Once the crimpable sleeve is in place, the sleeve 356 may be crimped or otherwise secured onto the extenders 354 in order to lock it in place. The excess material of the extenders 354 may be snipped off or otherwise removed. Although disclosed as extenders, the elements 354 may be tethers, ligaments, cables, threads, among other materials.

In other embodiments, the extenders are the deformable portion acting against the ligament to secure it in place, instead of the receiver itself. In such embodiments the receiver walls may be shorter than is shown in FIGS. 17A and B such that the extenders directly contact at least a portion of the ligament when in the final locking condition.

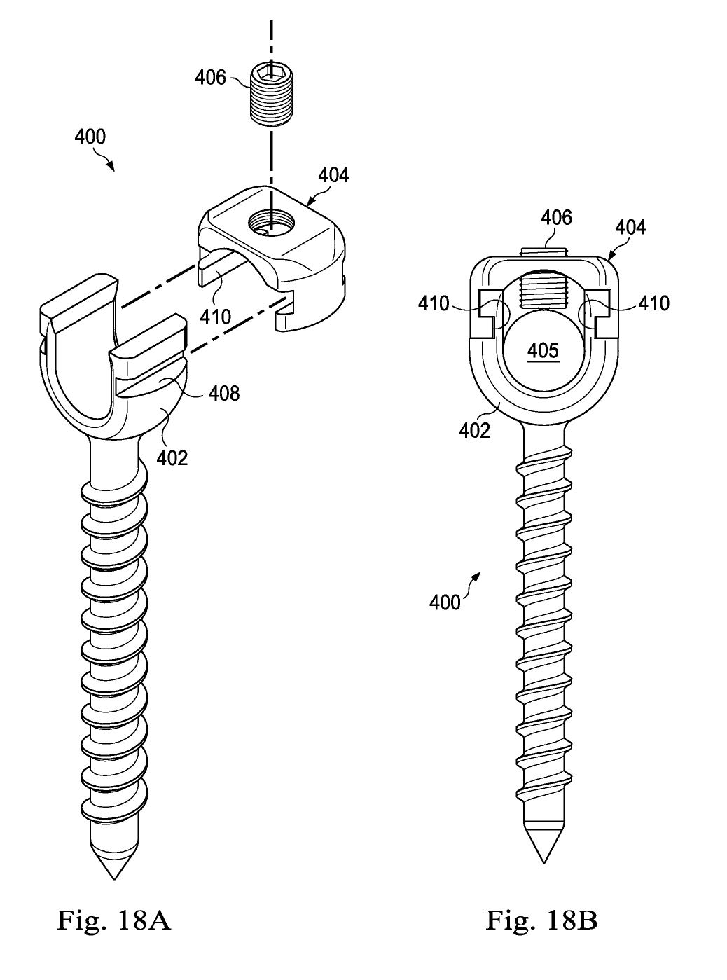

FIGS. 18A and 18B show another embodiment of a screw mechanism referenced herein by the numeral 400. Like the other embodiments described herein, the screw mechanism 400 includes a tulip-shaped receiver 402 for receiving ligament. In this embodiment, a locking system includes a staple 404 comprising a set screw 406. The staple 404 is receivable onto the receiver 402. In this embodiment, the staple 404 may be slid laterally such that grooves 408 formed in the exterior surface of the receiver 402 receive protrusions 410 formed in the inner surface of the staple 404 such that the staple 404 interlocks with the receiver 402.

The staple 404 includes an aperture 412 that receives the set screw 406. When the staple 404 is connected to the receiver 402, the ligament 405 is preliminarily locked within the screw mechanism 400 in that it can be further manipulated relative to the receiver 402. Tightening the set screw 406 against the ligament may finally lock the ligament relative to the screw mechanism 100. Although the embodiment shown in FIGS. 18A and 18B show a locking staple transversely connected to the receiver 402, in other embodiments, the staple connects to the receiver by advancing the staple over the receiver longitudinally as is shown in FIG. 5C. Although shown with a flat bottom, the set screw may have any bottom surface, including a central protrusion, knurling, or other configuration.

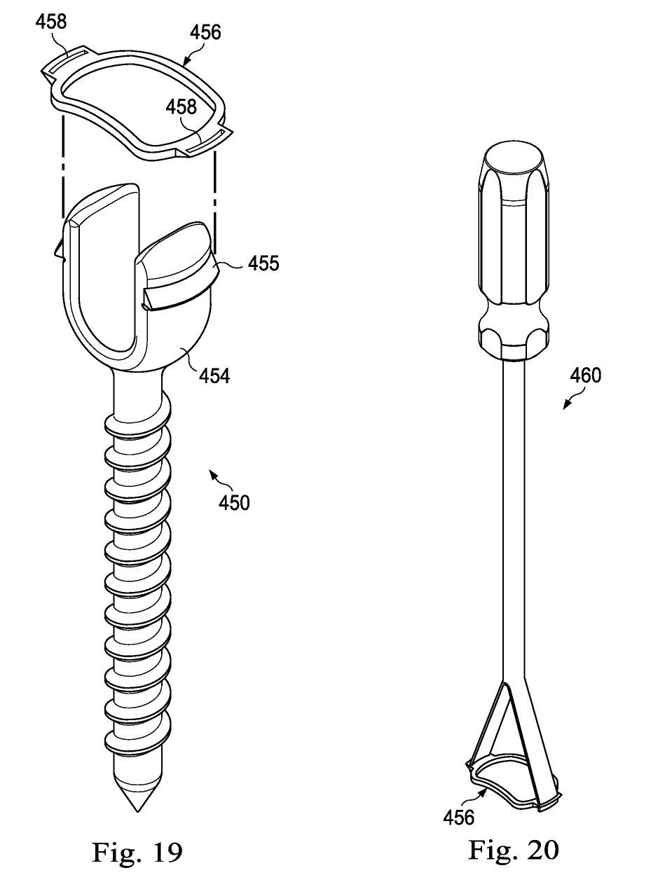

FIGS. 19-21 disclose an additional screw mechanism 450 that may be used to preliminarily lock and finally lock a ligament in place. This embodiment comprises a tulip head receiver 454 and a staple 456. The staple 456 is sized and shaped to extend about the exterior surface of the receiver 454. The external surface of the tulip head 454 includes at least one lip or edge 455 for snap-fitting or ratcheting the staple 456 onto the receiver 454. In this embodiment, the staple 456 is formed as a band with ears 458 that interface with an insertion tool 460 shown in FIG. 20. The tool 460 holds the staple 456 and connects the staple 456 to the receiver 454.

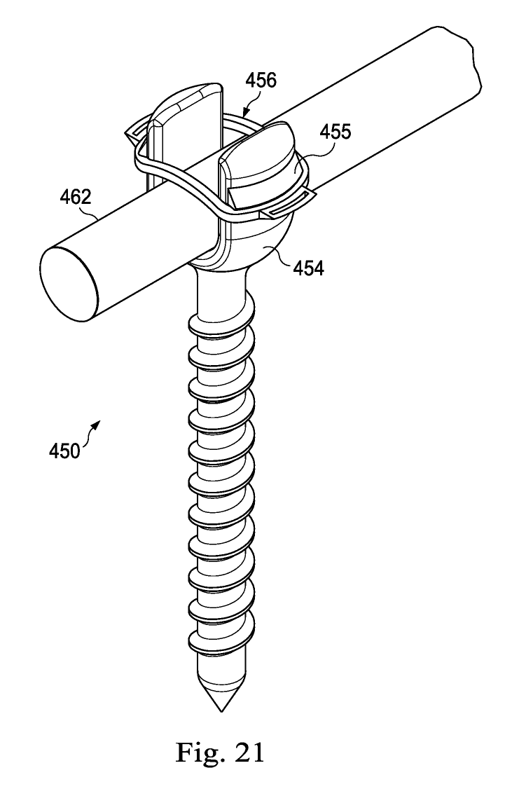

FIG. 21 shows the receiver head 454 with a ligament 462 contained therein. As can be seen, the staple 456 is secured about the circumference of the receiver below the lip or edge 455. In this manner the ligament 462 is secured in a final locking condition in the screw mechanism 450. In this embodiment, one or both of the staples 456 and the receiver 454 may be formed of a relatively elastic material that permits the staple 456 to be slid over the lip 455. Some embodiments include more than one edge or lip 455. In these embodiments, the staple 456 may be advanced over the first lip or edge while still leaving the ligament 462 relatively loose within the screw mechanism 450 for additional manipulation. To place the staple in a final locking condition, the staple may be further advanced over the additional tapered lock mechanisms until the staple compresses and secures the ligament within the receiver.

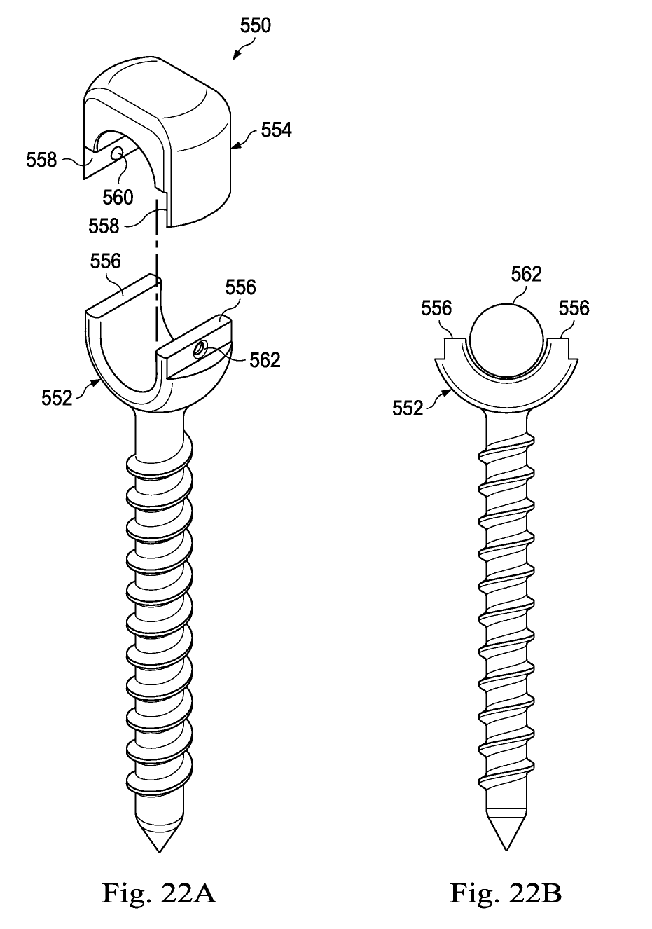

FIGS. 22A and 22B show another screw mechanism referenced herein by the numeral 550. Like the screw mechanisms described above, the screw mechanism 550 includes a tulip shaped receiver 552. A locking system for securing a ligament within the receiver 552 includes a staple 554 receivable over the receiver 552. In this embodiment, the receiver 552 includes legs 556 having a lower overall height than those in prior embodiments. For example, legs 556 extends from a base surface of a transverse opening formed by the tulip head a distance less than screw mechanisms described above. The staple 554, however, compensates for the short receiver by having legs 558 extending a length greater than the staples described above. Accordingly, although the overall height of the receiver 552 and staple 554 may be unchanged from prior embodiment, in this embodiment, the receiver legs 556 are shorter while the staple legs 558 are longer.

Together, the receiver legs 556 and the staple legs 558 form the transverse opening. In this embodiment, at last half of the circumference of the opening formed by the legs 556 and 558 is formed by the staple 558. In some embodiments, the staple forms between 50% and 80% of the circumference of the transverse opening formed by the staple legs and receiver legs. In some embodiments, the height of the legs 556 is less than the diameter of an associated ligament or rod. In some embodiments, the height of the legs 556 is less than one half of the diameter of an associated ligament or rod.

Accordingly, when a ligament 562 is placed within the receiver 552, the ligament 562 extends above the top of the tulip legs 556, or alternatively may fit neatly within the tulip leg 556 and the staple 554 provides the locking mechanism. In this embodiment, a connecting mechanism connects the receiver 552 and the staple 554. It includes a protrusion 560 receivable within a recess 562. As shown, the protrusion 560 is formed on the staple 554 and the recess 562 is formed on the receiver 552. In this embodiment, the staple 554 may be snapped into position via an introducer. This holds the ligament within the tulip of the receiver 552 that allows longitudinal sliding for tensioning under reductive maneuvers. In some embodiments, the staple includes an aperture that receives a set screw for securing the ligament similar to that shown in FIGS. 18A and 18B.

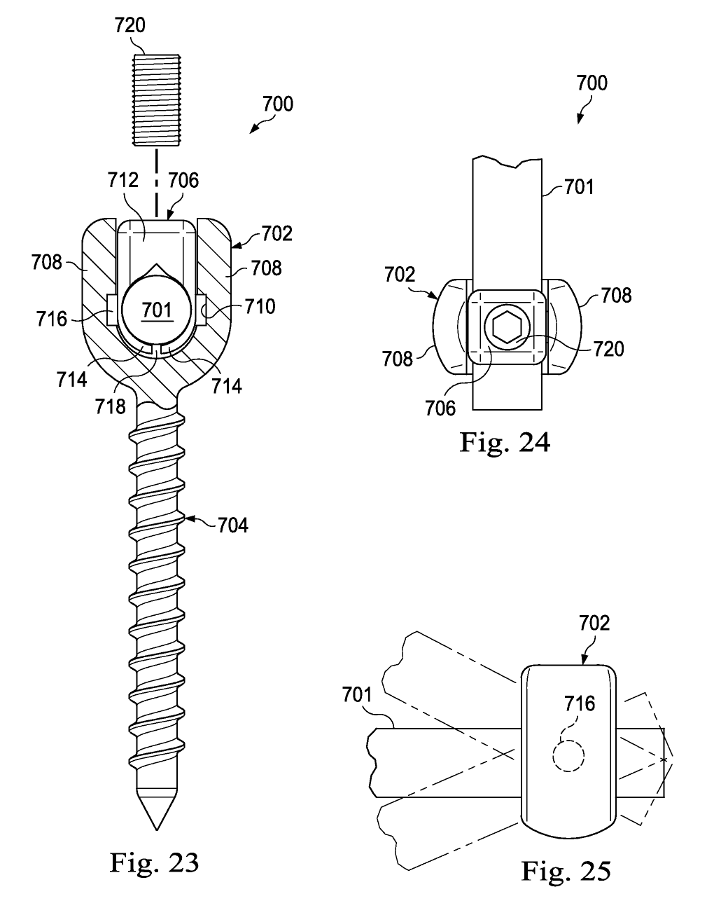

FIGS. 23-27 show a screw mechanism having a univariable head assembly that permits rotation of the ligament or a solid longitudinal member relative to the screw within at least a single plane. In this embodiment, the screw mechanism, referred to herein by the reference 700, includes a receiver 702, a threaded anchor portion 704, and an inner receiver referred to herein as an inner block 706 that provides variation in rod angle relative to the threaded anchor portion 704 and the receiver 702. In this embodiment, the inner block 706 is sized and configured to be disposed within the receiver 702.

The receiver 702, as in other embodiments disclosed herein, comprises two legs 708 forming a tulip-shaped head. Instead of directly interfacing with the ligament or rod, however, the receiver 702 receives the inner receiver as the inner block 706. In this embodiment, the receiver legs 708 each include a receiving depression 710 formed therein that receives corresponding portions of the inner block 706.

The inner block 706 includes a body 712 having legs 714 extending therefrom. The legs have articulating connectors 716 outwardly projecting therefrom that are receivable into the receiving depressions 710 of the receiver 702. In this embodiment, the articulating connectors 716 are cylindrical protrusions that fit within the receiving depressions 710 and are configured to articulate about an axis formed by the cylindrical shape. In this manner, the inner block 706 pivots about the articulating connectors 716 relative to the receiver 702. As shown in FIG. 23, when the inner block 706 is received within the receiver 702, the legs 714 are spaced from a bottom portion of the receiver 702. This is a preliminary locking condition, however, as the final locking condition will be discussed further below. The inner legs 714 are separated by a gap 718. The legs 714 and the body 712 together comprise a transverse opening sized and shaped to receive the rod 701. The body 712 includes a longitudinal opening for receiving a set screw 720. The longitudinal opening is in communication with the transverse opening.

FIG. 24 shows a top view of the screw mechanism 700. As can be seen, the set screw 720 is received within the inner block 706. It advances through an aperture formed in the top of the inner block 706 to directly engage and press against the rod 701. FIG. 25 is one example showing the articulation that may occur about the articulating connectors 716 within the receiving depressions 710. Accordingly, the screw mechanism 700 in FIGS. 23 through 26 permits rod angulations within a single plane. When the rod 701 extends through the transverse opening within the inner block 706, the rod 701 is preliminarily locked to the screw mechanism 700. It may still be advanced further through the opening or otherwise manipulated.

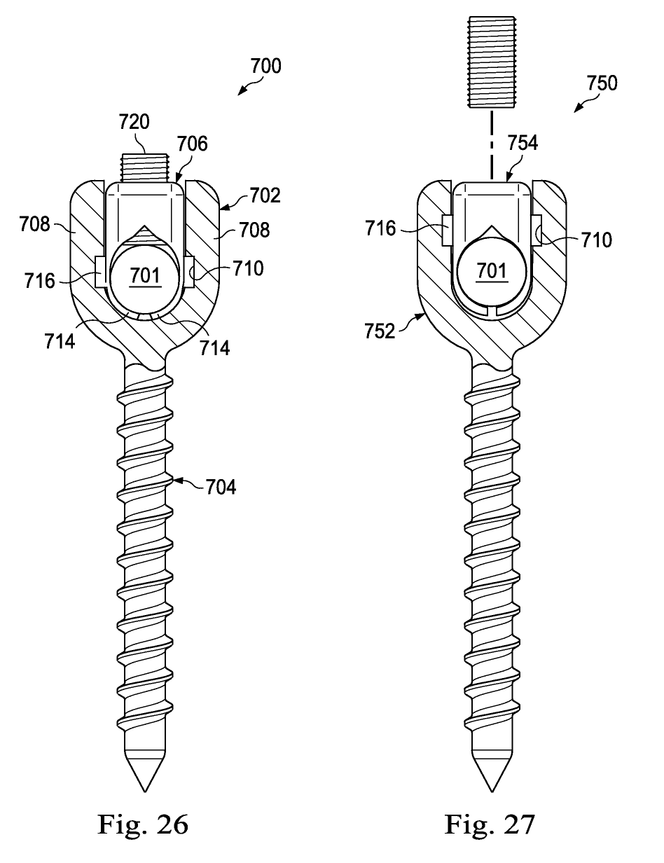

FIG. 26 shows the screw mechanism 700 in a final locking condition As can be seen, the set screw 720 is advanced until it contacts the rod 701. Further driving the set screw 720 displaces the rod 701 in the longitudinal direction of the screw mechanism 700. As the rod displaces, the rod acts against the legs 714 of the inner block 706 forcing them to splay apart into contact with the inner surface of the receiver 702. As the set screw 720 is further advanced, the legs 714 frictionally lock against the receiver 702 in a final locking condition, where the inner block 706 is immovable relative to the receiver 702. In this manner, the screw mechanism 700 has a provisional locking condition and a final locking condition for securing the rod 701 at a desired angled condition relative to the threaded anchor portion 704.

FIG. 27 shows another embodiment of a screw mechanism referenced herein as 750. The screw mechanism 750 is similar in most respects to the screw mechanism 700 just described and includes both a receiver 752 and an inner body 754. In this embodiment, however, the articulating connectors 716 and the receiving depression 710 formed within the receiver 752 are displaced from a lower portion of the receiver body to a higher area. Accordingly, a pivot axis of the inner block 754 relative to the receiver 754 does not intersect the longitudinal axis of the rod 701. Instead, in the embodiment shown, the pivot axis is spaced from or offset from the longitudinal axis of the rod 701. Accordingly, pivoting the inner block 754 relative to the receiver 752 results in a displacement more suitable for certain applications.

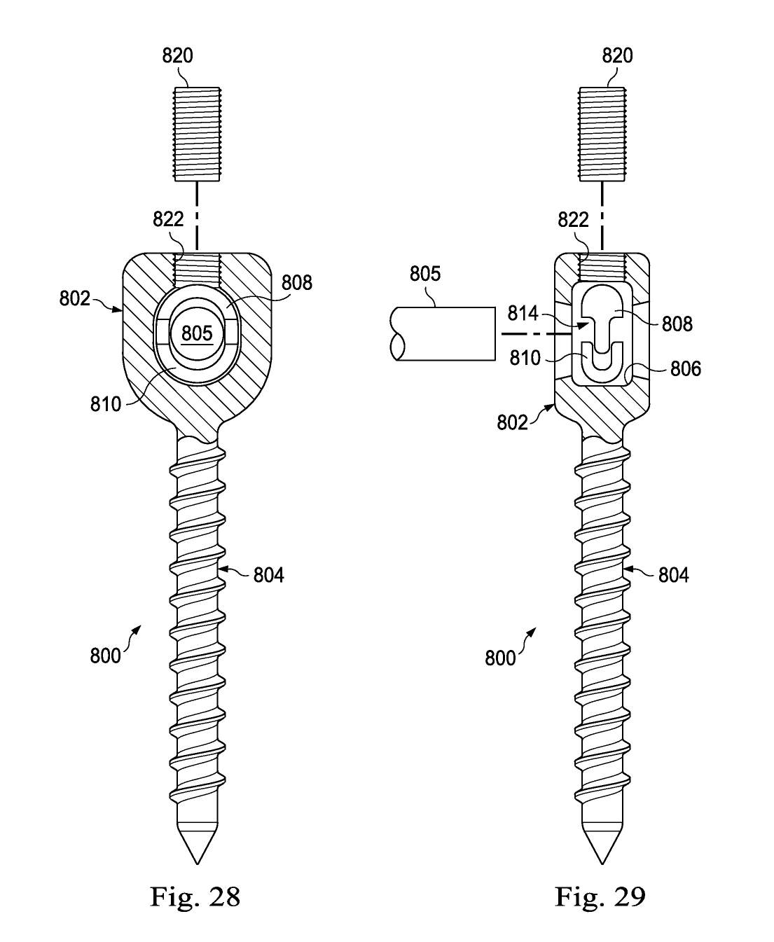

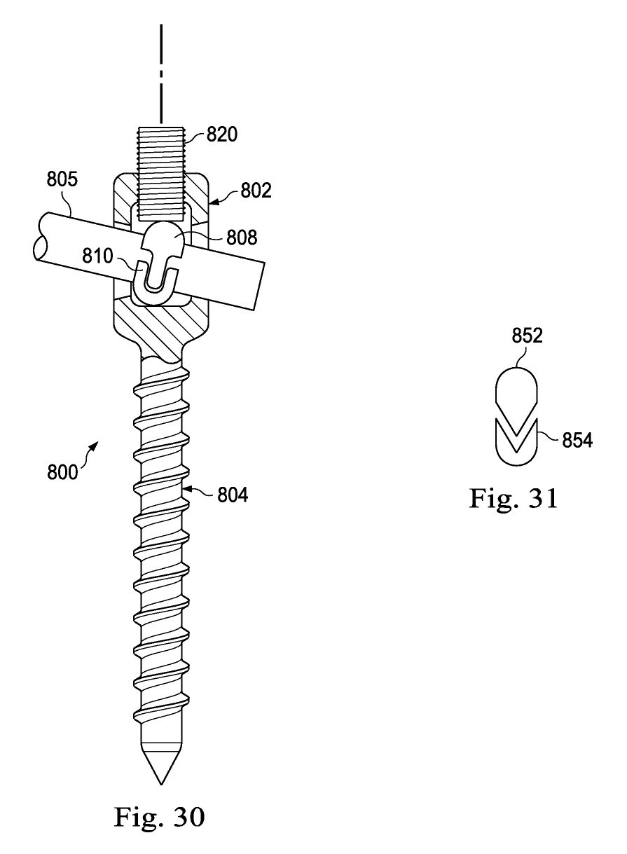

FIGS. 28 to 31 disclose a variable closed head connector screw mechanism referenced herein by the numeral 800. The screw mechanism 800 includes a receiver 802 and a threaded anchor portion 804. In this embodiment, the receiver 802 is a closed head receiver having openings to a transverse passage for receiving a rod 805. The receiver 802 includes a concave inner surface portion 806 forming a chamber that permits articulation of inner shells, or an inner receiver. In this embodiment, the receiver 802 forms an outer frame for an upper inner shell 808 and a bottom inner shell 810. These shells cooperate to form a ring around the rod 805 within the closed head receiver 802. In this embodiment, the rod 805 is spaced from the inner surface 806 of the receiver by the inner shells 808, 810. As best seen in FIG. 29, the shape of the concave surface 806 within the receiver 802 constrains movement and pivoting of the shells 808 and 810. In the embodiment shown, the range of articulation may be within the range of about 45°.

As shown in FIG. 29, the inner shells 808, 810 have interlocking portions 814 that aid in maintaining the shells connected together. Here, the interlocking portion 814 comprises a receiving U-shaped portion on the bottom inner shell 810 and a protruding tongue-shaped portion on the upper inner shell 808. The interlocking portions 814 are arranged to mechanically secure the inner shells together within the closed head receiver 802.

When a rod extends through the shells 808, 810, it can be manipulated with the shells 808, 810 within the receiver 802 until movement is locked using a locking system. Accordingly, the device has a preliminary locking condition. The rod can be held in a final locking condition by driving a set screw 820 through a passage 822 in the receiver 802. The set screw 820 contacts and bears against the inner shells 808, 810 to drive them together about the rod 805. This secures the rod in place relative to the shells and secures the shells 808, 810 into frictional engagement with the inner surface 806 of the closed head receiver 802. Accordingly, the set screw locks the screw mechanism 800 and the rod relative to the receiver 802. In some embodiments, even when the set screw is tightened, shell rotation is limited not only by the frictional engagement, but also by the shape of the concave surfaces 806. Accordingly, even when tightened about a rod, the shells may be sized to provide only a given range of articulation, which could be, for example, within a range of about 45 degrees. Other ranges also are contemplated, including 30 degrees, 60 degrees, and others.

FIG. 31 shows another set of inner shells 850, 852. Like those described above, these form a ring that can receive a rod. Here, instead of forming a rounded tongue and groove, the interlocking portions of the shells 850, 852 have a triangular-shape. As described above, one of the inner shells 850, 852 is received within the other inner shell to limit the movement of the inner shells relative to each other.

Applicants note that the use of directional terms herein, such as upper, lower, lateral, and others are merely exemplary, and may encompass other directions, such as the device being on its side, unless so indicated. Although several selected embodiments have been illustrated and described in detail, it will be understood that they are exemplary, and that a variety of substitutions and alterations are possible without departing from the spirit and scope of the present invention, as defined by the following claims.

1. A screw mechanism for connection with an elongated supporting structure, comprising:
a threaded anchor portion configured to penetrate bone tissue;
an outer receiver attached to the threaded anchor portion, the outer receiver being formed with first and second extending legs and having a transverse passage therethrough, the passage having inner sidewalls;
an inner receiver receivable into the outer receiver and having outer sidewalls, the outer sidewalls of the inner receiver and the inner sidewalls of the outer receiver passage having articulating or deformable connectors formed thereon, the connectors cooperating to permit the inner receiver to articulate relative to the outer receiver, the inner receiver being formed with a first longitudinally extending opening and having a second transverse opening therethrough sized and shaped to receive the stabilizing supporting structure, the inner receiver being deformable from an unlocked condition where the inner receiver is not frictionally engaged with the U-shaped inner surface in a manner that prevents articulation of the inner receiver relative to the outer receiver to a locked condition where the inner receiver is frictionally engaged with the U-shaped inner surface in a manner that prevents articulation of the inner receiver relative to the outer receiver;
a locking mechanism receivable into the first longitudinally extending opening and configured to apply a driving force that deforms the inner receiver from the unlocked condition to the locked condition.

2. The screw mechanism of claim 1, wherein the articulation occurs in a manner that the support structure is pivotable substantially along a single plane.

3. The screw mechanism of claim 1, wherein the first longitudinal opening of the inner receiver is in communication with the second transverse opening.

4. The screw mechanism of claim 1, wherein the inner receiver comprises legs that form at least a portion of the second transverse opening, the legs being deformable from the unlocked condition where the legs are not frictionally engaged with the U-shaped inner surface in a manner that prevents articulation of the inner receiver relative to the receiver to the locked condition where the legs are frictionally engaged with the U-shaped inner surface in a manner that prevents articulation of the inner receiver relative to the outer receiver.

5. The screw mechanism of claim 4, wherein the legs of the inner receiver are shaped to define a gap therebetween when in the locked condition.

6. The screw mechanism of claim 5, wherein the gap is centrally disposed along the longitudinal axis.

7. The screw mechanism of claim 4, wherein the legs are arranged to splay when the set screw is driven against the elongated support structure.

8. The screw mechanism of claim 1, wherein the outer receiver legs form a U-shaped inner surface.

9. The screw mechanism of claim 1, wherein the first longitudinally extending opening includes threads on an inner circumference and wherein the locking mechanism is a set screw.

10. The screw mechanism of claim 1, wherein the articulating connectors are a receiving depression in the inner sidewalls of the outer receiver and protrusions in the outer sidewalls of the inner receiver received into the receiving depressions.

11. The screw mechanism of claim 1, wherein the articulating connectors are disposed at a location substantially transverse to the elongated supporting structure.

12. The screw mechanism of claim 1, wherein the articulating connectors define a pivot axis, the pivot axis being substantially transverse to a longitudinal axis of the elongated supporting structure.

13. The screw mechanism of claim 1, wherein the articulating connectors define a pivot axis that is offset from a longitudinal axis of the elongated supporting structure

14. The screw mechanism of claim 1, wherein the articulating connectors define a pivot axis that is offset from the elongated supporting structure.

15. A screw mechanism for connection with an elongated supporting structure, comprising:
a threaded anchor portion configured to penetrate bone tissue;
an outer receiver attached to the threaded anchor portion, the receiver being formed with first and second extending legs forming a U-shaped inner surface, the U-shaped surface having a bottom surface and inner sidewalls;
an inner receiver receivable into the outer receiver, the inner receiver having outer sidewalls, the inner receiver being formed with a first longitudinally extending opening having threads on the inner circumference and having a second transverse opening therethrough sized and shaped to receive the elongated supporting structure, the inner receiver having legs deformable from an unlocked condition where the legs are not frictionally engaged with the U-shaped inner surface in a manner that prevents articulation of the inner receiver relative to the outer receiver to a locked condition where the legs are frictionally engaged with the U-shaped inner surface in a manner that prevents articulation of the inner receiver relative to the outer receiver;
a locking mechanism receivable into the first longitudinally extending opening and configured to apply a driving force that deforms the inner receiver from the unlocked condition to the locked condition.

16. The screw mechanism of claim 15, wherein the articulation occurs in a manner that the support structure is pivotable substantially along a single plane.

17. The screw mechanism of claim 15, wherein the legs are arranged to splay when the set screw is driven against the elongated support structure.

18. The screw mechanism of claim 15, wherein the first longitudinally extending opening includes threads on an inner circumference and wherein the locking mechanism is a set screw.

19. The screw mechanism of claim 15, wherein the outer sidewalls of the inner receiver and the inner sidewalls of the U-shaped inner surface having articulating connectors formed thereon, the connectors cooperating to permit the inner receiver to articulate relative to the outer receiver and defining a pivot axis, the pivot axis being substantially transverse to a longitudinal axis of the elongated supporting structure.

20. A screw mechanism for connection with an elongated supporting structure, comprising:
a threaded anchor portion configured to penetrate bone tissue;
a receiver attached to the threaded anchor portion, the receiver having a transverse passage therethrough, the passage having inner sidewalls;
a locking system having a preliminary locking condition and a final locking condition, the preliminary locking condition permitting manipulation of the elongated supporting member within the transverse passage while preventing removal of the elongated supporting member, and the locking condition fixedly securing the elongated member in the transverse passage.

21. The screw mechanism of claim 20, wherein the receiver comprises a first arm and a second arm, the second arm being moveable relative to the first arm, the locking system comprising ratcheting teeth on one of the first and second arms.

22. The screw mechanism of claim 21, wherein the receiver comprises a slot or aperture for receiving at least a portion of one of the first and second arms, the locking mechanism further comprising a cap rotatable to place the locking system in one of the preliminary locking condition and the final locking condition.

23. The screw mechanism of claim 20, wherein the locking system comprises:
a plurality of shells disposable about the elongated support member and compressible between the preliminary locking condition and the final locking condition; and a locking cap that cooperates with the shells to place the locking system in the final locking condition.

24. The screw mechanism of claim 20, wherein the locking system comprises a sleeve portion crimpable to place the locking system in the final locking condition.

25. The screw mechanism of claim 20, wherein the locking system comprises a cap portion fittable over a portion of the receiver, at least one of the cap portion and the receiver being deformable so that the cap snaps onto the receiver.

26. The screw mechanism of claim 20, wherein the locking system comprises a cap portion engagable with the receiver via in interlocking fit, the cap portion including an aperture with a set screw therein.

27. The screw mechanism of claim 20, wherein the locking system comprises a staple, the staple fitting over at least a portion of an exterior surface of the receiver in a manner that the staple snaps onto the receiver.

28. The screw mechanism of claim 27, wherein one of the staple and receiver comprises at least one of a shoulder, a lip, and a protrusion that secures the staple and receiver in the final locking condition.

29. The screw mechanism of claim 20, wherein the receiver comprises a chamber formed therein, the locking system comprising a plurality of shells disposed within the chamber and clampable about the elongated support structure.
